(19) United States
(12) Patent Application Publication
(10) Pub. No.: US 2010/0105880 A1
(43) Pub. Date: Apr. 29, 2010

(54) PURIFICATION OF CARBON NANOTUBES VIA BIOMOLECULES

(75) Inventor: LETHA J. SOOTER, Morgantown, WV (US)

Correspondence Address: U S ARMY RESEARCH LABORATORY;ATTN: RDRL-LOC-I, 2800 POWDER MILL RD, ADELPHI, MD, 20783-1197, US

(73) Assignee: UNITED STATES OF AMERICA AS REPRESENTED BY THE SECRETARY OF THE ARMY

(21) Appl. No.: 12/571,098

(22) Filed: Sep. 30, 2009

Related U.S. Application Data
(60) Provisional application No. 61/136,767, filed on Oct. 1, 2008.

(51) Int. Cl. D01F 9/12; B01J 8/02; C07K 17/14; C07C 229/00; C07H 19/00; C07H 21/00

(52) U.S. Cl. 530/402; 422/212; 423/447.1; 562/442; 536/22.1; 536/23.1; 977/845; 977/742

(57) ABSTRACT

Separating different types of nanotubes from one another includes providing a sample of heterogeneous nanotubes comprising a first and second type of carbon nanotube; providing a first type of molecule; introducing the first type of molecule to the sample; binding the first type of molecule to the first type of carbon nanotube; and separating the first type of carbon nanotube from the sample. A second type of molecule may be introduced to the sample followed by binding the second type of molecule to the second type of carbon nanotube; and separating the second type of carbon nanotube from the sample. The sample may comprise a third type of carbon nanotube. A third type of molecule may be introduced to the sample followed by binding the third type of molecule to the third type of carbon nanotube; and separating the third type of carbon nanotube from the sample.

### CROSS REFERENCE TO RELATED APPLICATION

This application claims the benefit of U.S. Provisional Patent Application No. 61/136,767 filed on Oct. 1, 2008, the complete disclosure of which is hereby incorporated by reference herein in its entirety.

### GOVERNMENT INTEREST

The embodiments herein may be manufactured, used, and/or licensed by or for the United States Government without the payment of royalties thereon.

### TECHNICAL FIELD

The embodiments described herein generally relate to carbon nanotube technology and, more particularly, to the selection and separation of carbon nanotubes.

### BACKGROUND

It would be desirable to select and separate specific carbon nanotubes from a population of carbon nanotubes and other mixtures that includes a mixture of carbon nanotubes. For example, carbon nanotubes may be selected and separated using biomolecules based on one or more of the following characteristics: single wall nanotubes versus multiwall nanotubes; varying degrees of multiwall nanotubes, such as biwall versus triwall nanotubes; differing chiral vectors; differing translational vectors; differing symmetry vectors; and even differing molecular weights, sizes or geometries.

Currently it is extremely difficult to isolate uniform carbon nanotube populations. This hinders innovation due to the non-optimal starting material. Manufacturers that are interested in using carbon nanotubes, for example to make lighter materials, stronger materials or materials or products with other improved properties, would benefit from using more uniform carbon nanotube starting materials in their products and processes.

### SUMMARY

In view of the foregoing, an embodiment herein provides a method of purifying nanotubes, the method comprising providing a sample of heterogeneous nanotubes comprising at least two different types of carbon nanotubes comprising a first type of carbon nanotubes and a second type of carbon nanotubes; providing a plurality of first type of molecules; introducing the sample of heterogeneous nanotubes to the plurality of first type of molecules; binding the first type of carbon nanotubes to the plurality of first type of molecules; and separating the first type of carbon nanotubes from the sample.

The method may further comprise providing a plurality of second type of molecules; introducing the sample of heterogeneous nanotubes to the plurality of second type of molecules; binding the second type of carbon nanotubes to the plurality of second type of molecules; and separating the second type of carbon nanotubes from the sample. Moreover, the at least two different types of carbon nanotubes may comprise a third type of carbon nanotubes. The method may further comprise providing a plurality of third type of molecules; introducing the sample of heterogeneous nanotubes to the plurality of third type of molecules; binding the third type of carbon nanotubes to the plurality of third type of molecules; and separating the third type of carbon nanotubes from the sample.

The method may further comprise washing the sample of heterogeneous nanotubes. Additionally, the method may further comprise washing the first type of molecules, the second type of molecules, and the third type of molecules. Also, any of the first type of molecules, the second type of molecules, and the third type of molecules may comprise any of a peptide, protein, amino acid, nucleic acid, deoxyribonucleic acid, ribonucleic acid, and peptide nucleic acid. Furthermore, the first type of carbon nanotubes, the second type of carbon nanotubes, and the third type of carbon nanotubes are preferably each less than approximately 40 microns in length.

Another embodiment provides a method of separating different types of nanotubes from one another using non-chemical processing, the method comprising providing a sample of heterogeneous nanotubes comprising a first type of carbon nanotube and a second type of carbon nanotube; providing a first type of molecule; introducing the first type of molecule to the sample of heterogeneous nanotubes; binding the first type of molecule to the first type of carbon nanotube; and separating the first type of carbon nanotube from the sample. The method may further comprise providing a second type of molecule; introducing the second type of molecule to the sample of heterogeneous nanotubes; binding the second type of molecule to the second type of carbon nanotube; and separating the second type of carbon nanotube from the sample.

Moreover, the sample of heterogeneous nanotubes may comprise a third type of carbon nanotube. The method may further comprise providing a third type of molecule; introducing the third type of molecule to the sample of heterogeneous nanotubes; binding the third type of molecule to the third type of carbon nanotube; and separating the third type of carbon nanotube from the sample. Additionally, the method may further comprise washing any of the sample of heterogeneous nanotubes, and the first type of molecule, the second type of molecule, and the third type of molecule. Also, any of the first type of molecule, the second type of molecule, and the third type of molecule may comprise any of a peptide, protein, amino acid, nucleic acid, deoxyribonucleic acid, ribonucleic acid, and peptide nucleic acid, and wherein the first type of carbon nanotube, the second type of carbon nanotube, and the third type of carbon nanotube are preferably each less than approximately 40 microns in length.

Another embodiment provides an apparatus for purifying nanotubes, the apparatus comprising a sample of heterogeneous nanotubes comprising at least two different types of carbon nanotubes comprising a first type of carbon nanotubes and a second type of carbon nanotubes; a purification column comprising at least one solid support structure comprising a plurality of first type of molecules; a buffer flow zone in the purification column that allows the sample of heterogeneous nanotubes to interact with the plurality of first type of molecules, wherein the interaction comprises (i) binding the first type of carbon nanotubes to the plurality of first type of molecules, and (ii) separating the first type of carbon nanotubes from the sample; and a first collection unit that collects the separated the first type of carbon nanotubes.

Furthermore, the at least one solid support structure may comprise a first solid support structure comprising the plurality of first type of molecules, wherein the apparatus may further comprise a second solid support structure comprising a plurality of second type of molecules, wherein the buffer flow zone allows the sample of heterogeneous nanotubes to interact with the plurality of second type of molecules, wherein the interaction comprises (iii) binding the second type of carbon nanotubes to the plurality of second type of molecules, and (iv) separating the second type of carbon nanotubes from the sample, and wherein the apparatus may further comprise a second collection unit that collects the separated the second type of carbon nanotubes.

Moreover, the at least two different types of carbon nanotubes may comprise a third type of carbon nanotubes. The apparatus may further comprise a third solid support structure comprising a plurality of third type of molecules, wherein the buffer flow zone allows the sample of heterogeneous nanotubes to interact with the plurality of third type of molecules, wherein the interaction comprises (v) binding the third type of carbon nanotubes to the plurality of third type of molecules, and (vi) separating the third type of carbon nanotubes from the sample, and wherein the apparatus may further comprise a third collection unit that collects the separated the third type of carbon nanotubes. Also, any of the first type of molecules, the second type of molecules, and the third type of molecules may comprise any of a peptide, protein, amino acid, nucleic acid, deoxyribonucleic acid, ribonucleic acid, and peptide nucleic acid. Additionally, the first type of carbon nanotubes, the second type of carbon nanotubes, and the third type of carbon nanotubes are preferably each less than approximately 40 microns in length.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a schematic diagram of a process of in vitro selection of biomolecules according to an embodiment herein;

FIG. 2 illustrates a schematic diagram of an apparatus for separating and purifying carbon nanotubes according to an embodiment herein;

FIG. 3 illustrates a schematic diagram of an isolated view of the apparatus of FIG. 2 according to an embodiment herein; and

FIGS. 4 through 10 illustrate schematic diagrams of the apparatus of FIG. 2 in sequential processing steps according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

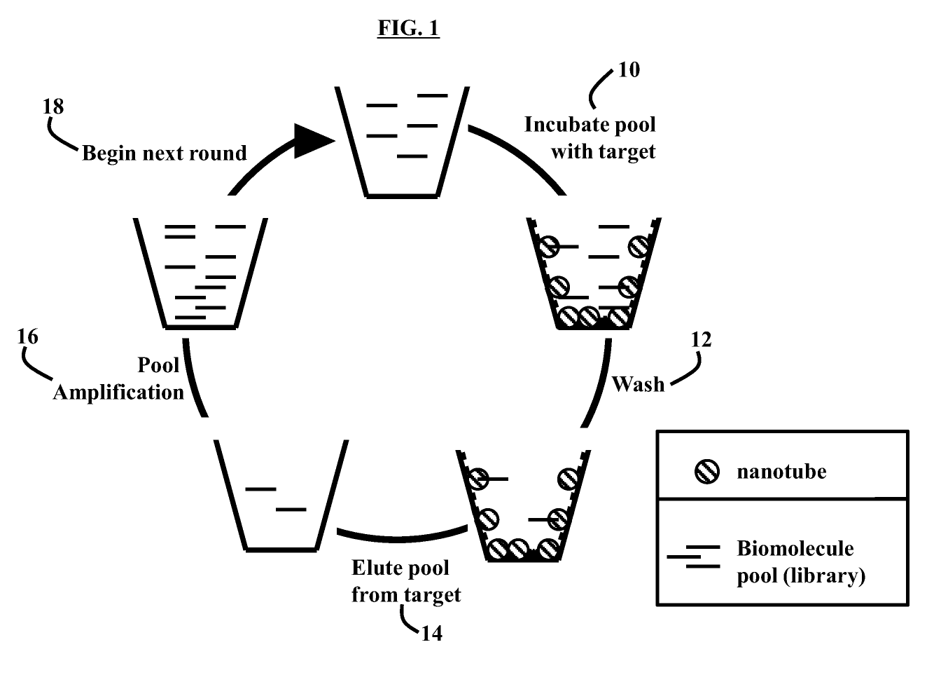

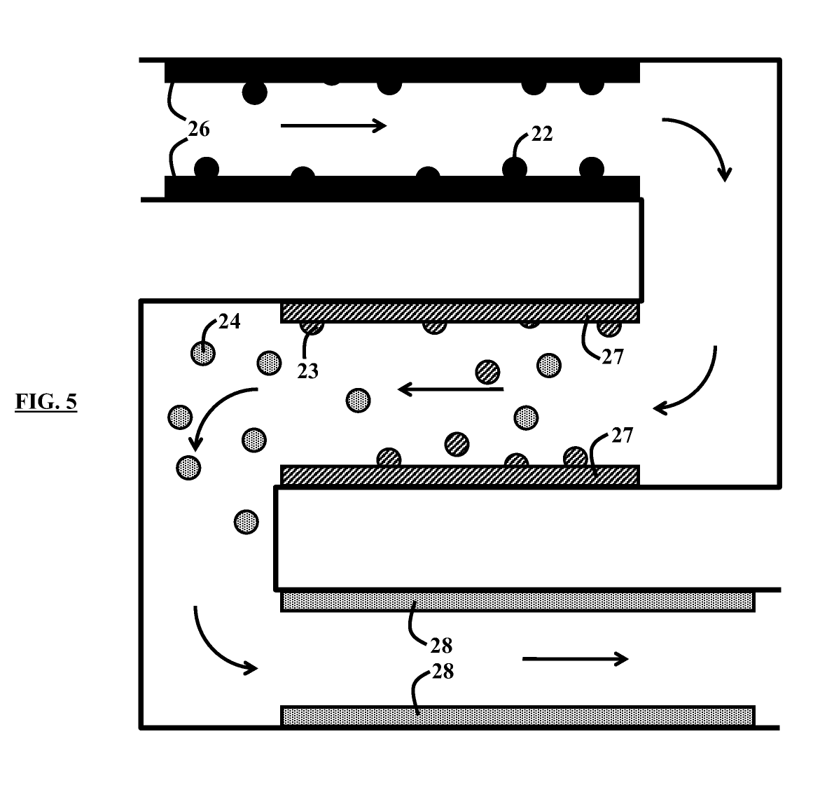

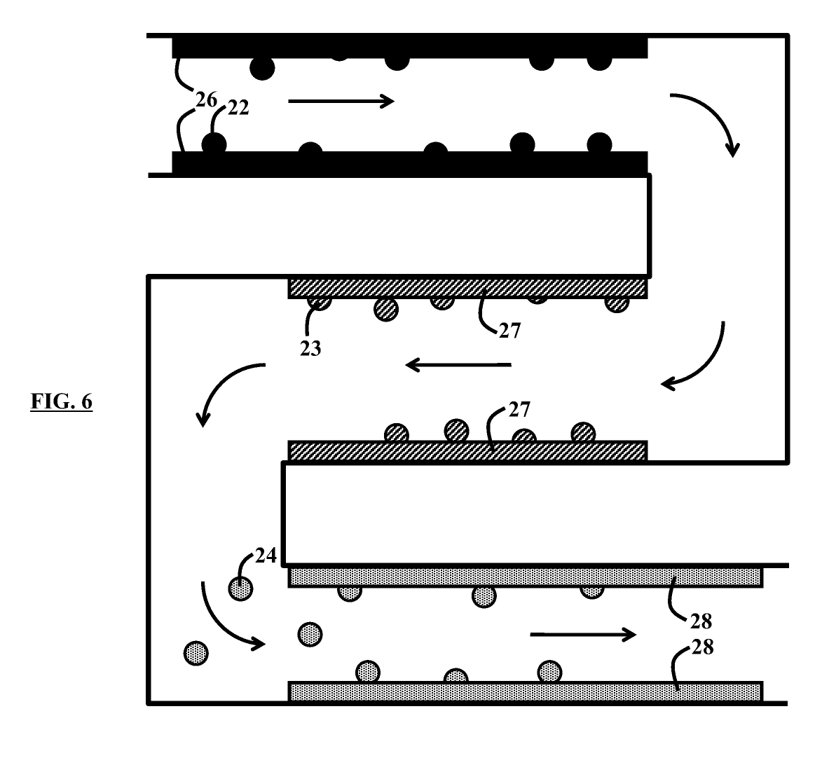

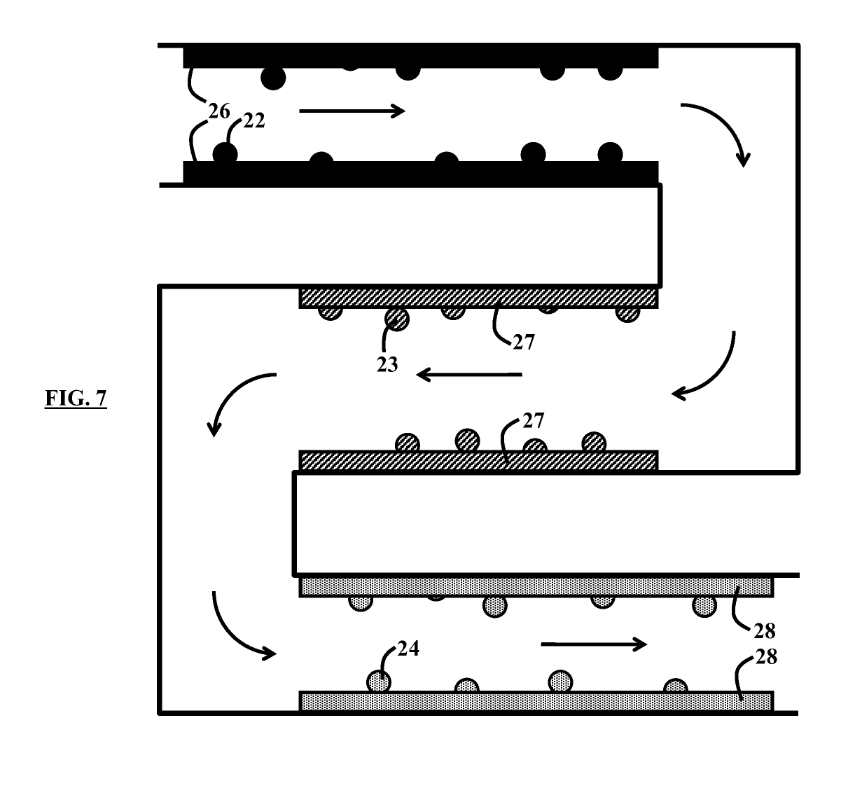

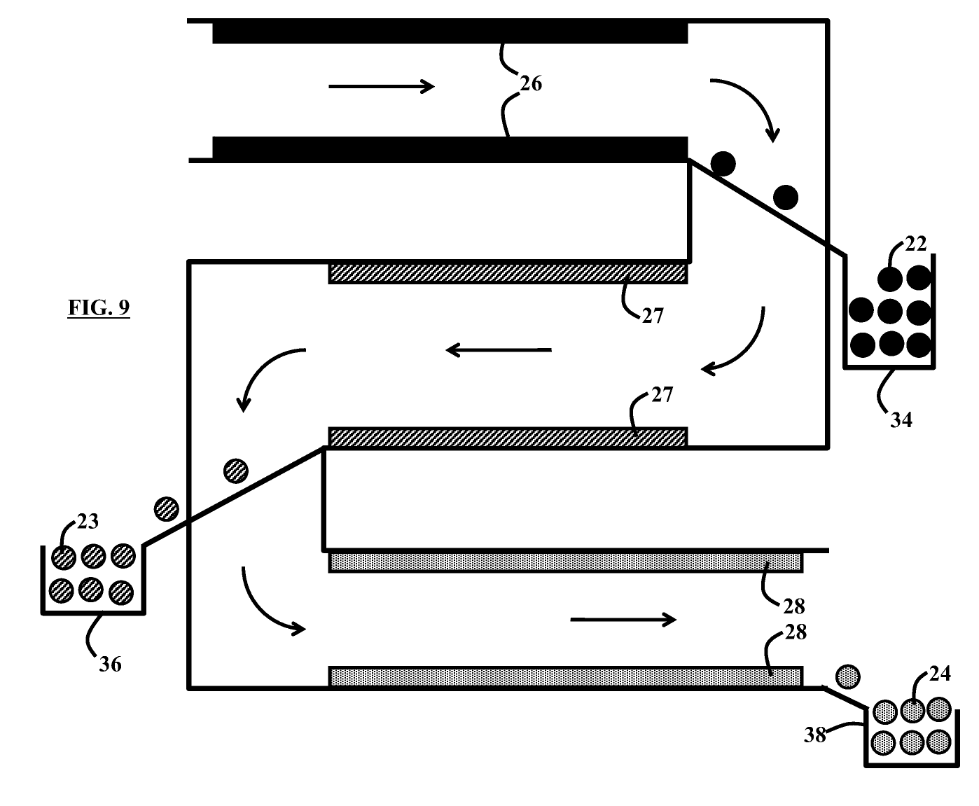

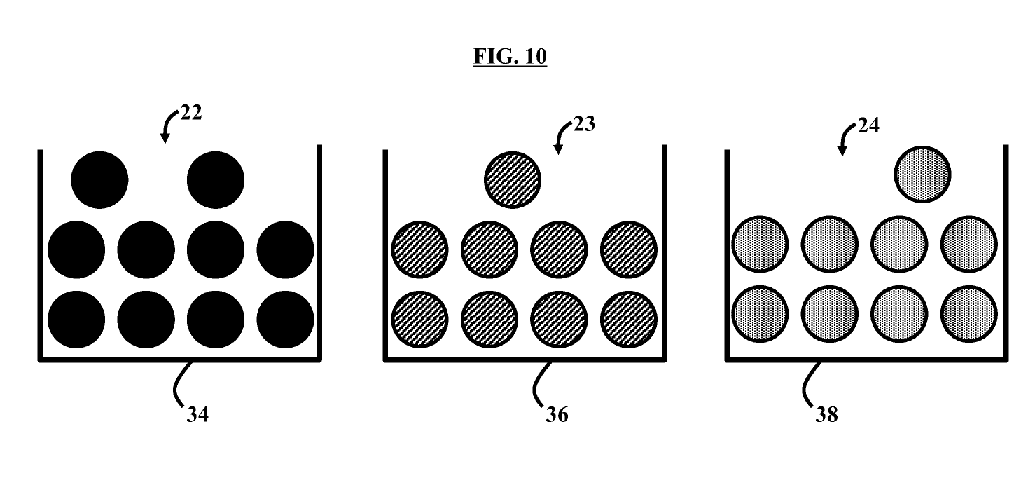

The embodiments described and illustrated herein provide for the differentiation, the selection, and/or the purification of different allotropes of carbon using biomolecules, and provides an apparatus and a method for selecting and separating different types and/or sizes of carbon nanotubes from a mixture containing different types and/or sizes of carbon nanotubes using biomolecules. Referring now to the drawings, and more particularly to FIGS. 1 through 10, where similar reference characters denote corresponding features consistently throughout the figures, there are shown preferred embodiments.

Carbon nanotubes may have different atomic compositions, different diameters, different chiralities, different defects, and be single or multi-wall. All of these characteristics give the carbon nanotubes different properties. Biomolecules may be composed of nucleic acids, nucleic acid derivatives, amino acids, and/or amino acid derivatives.

In vitro selections can be used to select biomolecules, for example proteins, peptides, amino acid, deoxyribonucleic acid (DNA), ribonucleic acid (RNA) and peptide nucleic acid (PNA), that bind nanotubes. These binders are able to differentiate between one or more of the following nanotube characteristics: single versus multi-wall, degrees of multi-wall, different chiral vectors, different translational vectors, and different symmetry vectors. The general process of in vitro selection of biomolecules for binding affinity and specificity is shown in FIG. 1. Nanotubes that are less than approximately 40 microns in length can be incubated in the presence of a fluorescently labeled library as indicated in step 10. The mixture can then be run/washed through a fluorescent activated cell sorting (FACS) machine as indicated in step 12. The bound and unbound nanotubes can be separated into two populations (elute biomolecule pool from target) as indicated in step 14. Then, the library fraction that is bound can be amplified and subjected to additional rounds of selection as indicated in step 16. The final binding species that are isolated bind very tightly and specifically. Moreover, the process can begin anew as indicated in step 18 with new a new biomolecule pool and nanotubes.

Peptides and nucleic acids may be synthesized on solid supports. The mixed nanotube population flows across the solid support which specifically binds a particular type of nanotube. This specific nanotube binds to the biomolecule on the solid support and the other nanotubes continue to flow past. The solid support is then washed, followed by the elution of a uniform nanotube population. Examples of elution methods include increased temperature or a change in the ionic strength of the solution. Following a step to regenerate the biomolecule binding structure, such as a heating and cooling step, the biomolecule coated support is be ready for another round of separation.

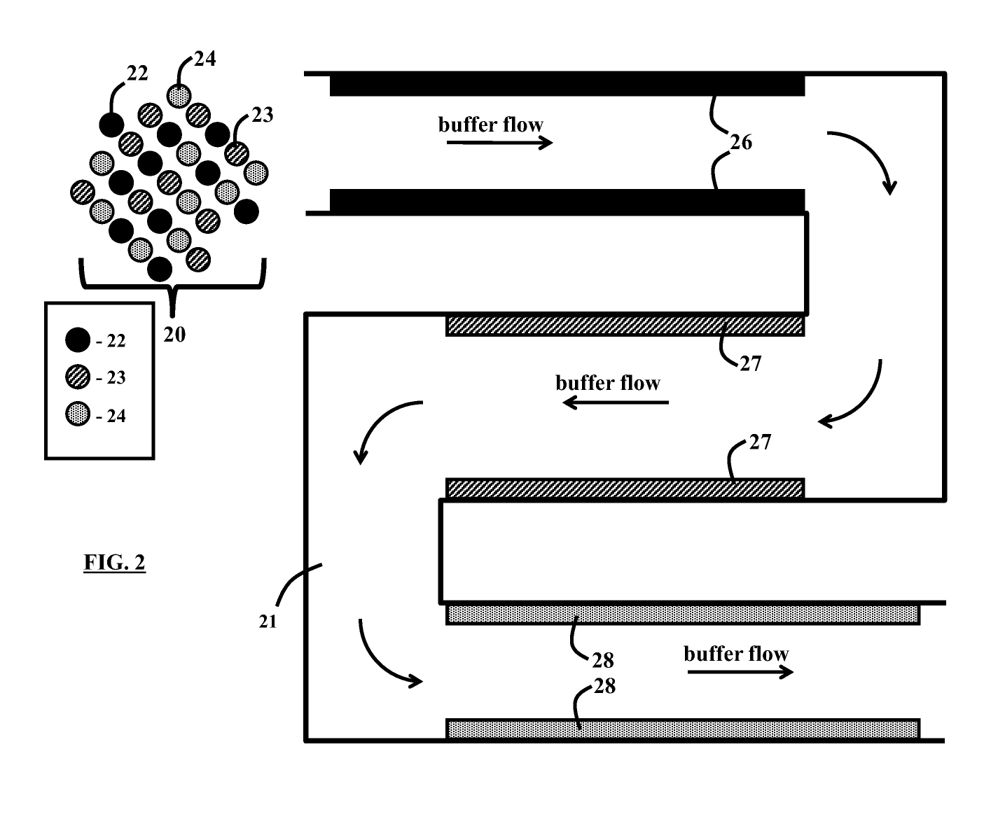

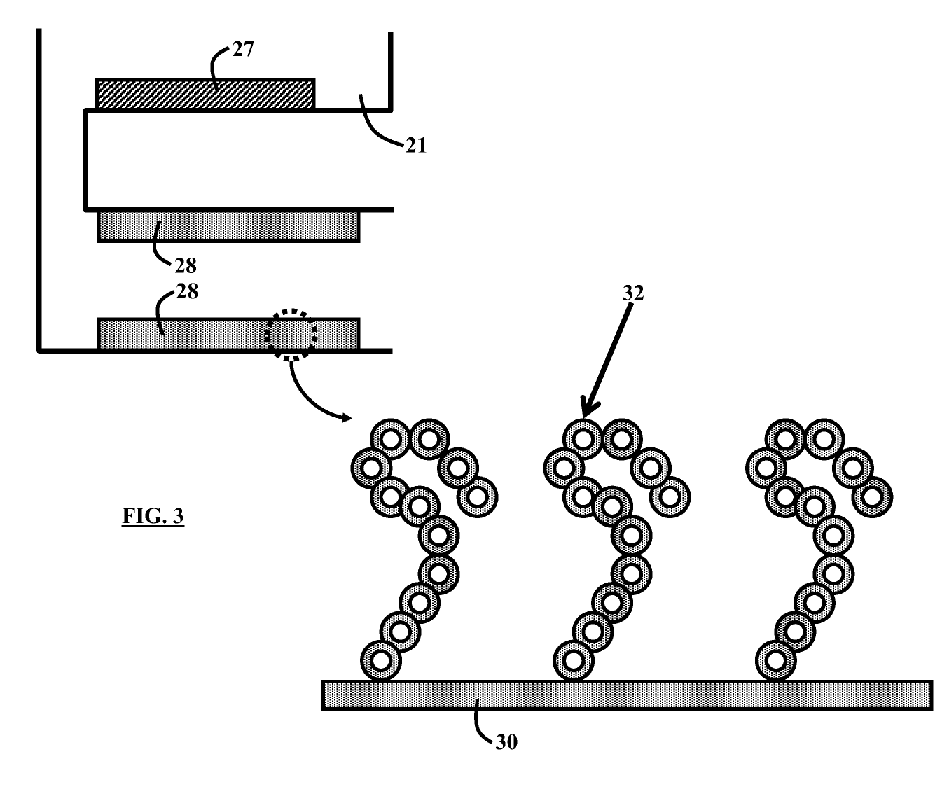

A process of purifying and separating a heterogeneous mixture of carbon molecules by using one or more biomolecules that bind to specific a nanotube is illustrated in FIGS. 2 through 10 and further described below. FIG. 2 illustrates an apparatus that is used in the purification process, whereby the process generally involves introducing a sample batch of heterogeneous nanotubes 20 into a purification column 21 configured to have a specified buffer flow. The purification column 21 comprises a plurality of biomolecules 26, 27, 28 that may be configured as a modified column wall comprising a solid support structure 30 with biomolecule chains 32 affixed thereto. Desirably, the biomolecule chains are selected so that the biomolecules bind to specific nanotubes with high affinity and specificity. FIG. 3 illustrates an example of a magnified view of the biomolecules 28. However, FIG. 3 is for illustrative purposes only, and the configuration shown in FIG. 3 is equally applicable to biomolecules 26 and 27 as well.

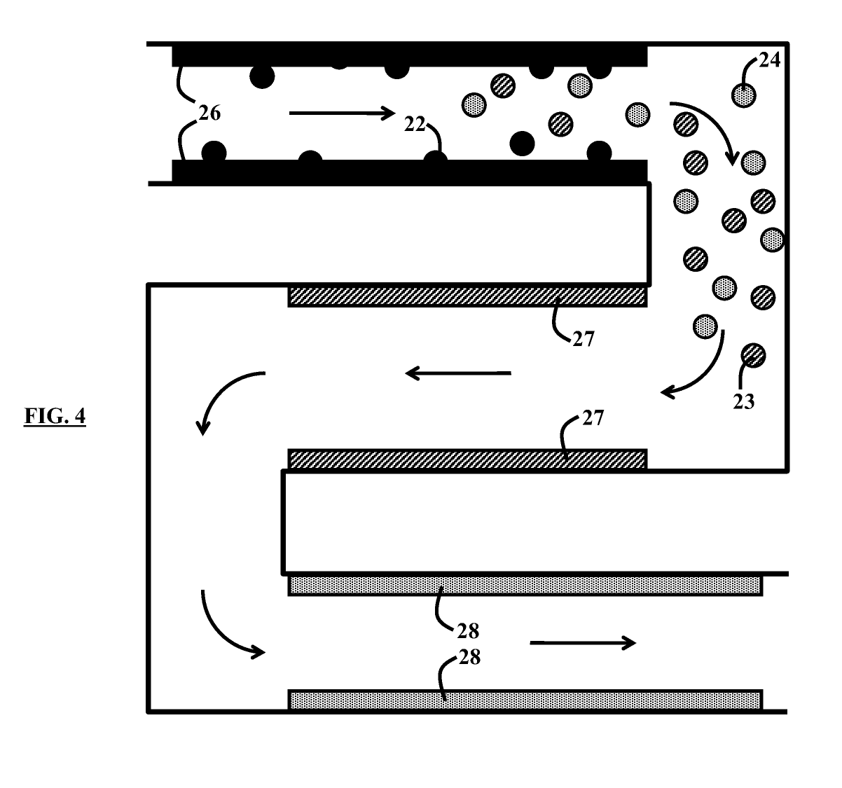

FIG. 2 illustrates providing a sample 20 that includes a mixture of at least three or more types of carbon nanotubes 22, 23, 24. FIGS. 4 through 7 illustrate providing a plurality of first molecules 26 that bind to the first type of carbon nanotubes 22, providing a plurality of second molecules 27 that bind to the second type of carbon nanotubes 23, and providing a plurality of third molecules 28 that bind to the third type of carbon nanotubes 24. Accordingly, the plurality of first molecules 26, the plurality of second molecules 27, and the plurality of third molecules 28 are introduced to the sample 20 that includes the mixture of at least three or more types of carbon nanotubes 22, 23, 24. An alternative embodiment is to introduce the molecules 26, 27, 28 to the sample 20 of carbon nanotubes 22, 23, 24. Then, at least a portion of the plurality of first molecules 26 bind to at least a portion of the first type of carbon nanotubes 22, at least a portion of the plurality of second molecules 27 bind to at least a portion of the second type of carbon nanotubes 23, and at least a portion of the plurality of third molecules 28 bind to at least a portion of the third type of carbon nanotubes 24.

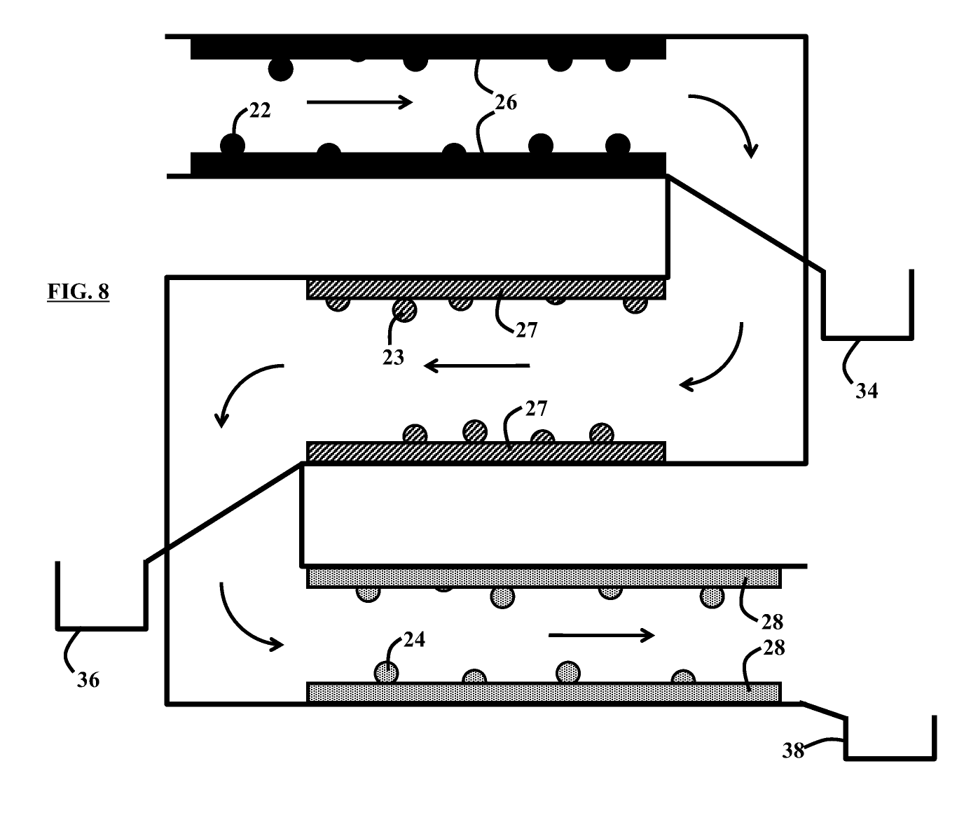

FIGS. 8 through 10, with reference to FIGS. 1 through 7, illustrate separating the portion of the plurality of first molecules 26 bound to at least a portion of the first type of carbon nanotubes 22 from the sample 20, separating the portion of the plurality of second molecules 27 bound to at least a portion of the second type of carbon nanotubes 23 from the sample 20, and separating the portion of the plurality of third molecules 28 bound to at least a portion of the third type of carbon nanotubes 24 from the sample 20 into corresponding collection units 34, 36, 38 resulting in purified nanotube batches.

In another embodiment, the process comprises providing a sample 20 that includes a mixture of at least two or more types of carbon nanotubes 22, 23; providing a plurality of first molecules 26 that bind to a first type of carbon nanotubes 22; providing a plurality of second molecules 27 that bind to a second type of carbon nanotubes 23; introducing the plurality of first molecules 26 and the plurality of second molecules 27 to the sample 20 that includes the mixture of at least two or more types of carbon nanotubes 22, 23; binding at least a portion of the plurality of first molecules 26 to at least a portion of the first type of carbon nanotubes 22, and binding at least a portion of the plurality of second molecules 27 to at least a portion of the second type of carbon nanotubes 23; separating the portion of the plurality of first molecules 26 bound to at least a portion of the first type of carbon nanotubes 22 from the sample 20; and separating the portion of the plurality of second molecules 27 bound to at least a portion of the second type of carbon nanotubes 23 from the sample 20.

In another embodiment, the method of nanotube purification comprises providing a sample 20 that includes a mixture of at least two or more types of carbon nanotubes 22, 23; providing a plurality of first molecules 26 that bind to a first type of carbon nanotubes 22; introducing the plurality of first molecules 26 to the sample 20 that includes the mixture of at least two or more types of carbon nanotubes 22, 23; binding at least a portion of the plurality of first molecules 26 to at least a portion of the first type of carbon nanotubes 22; and separating the portion of the plurality of first molecules 26 bound to at least a portion of the first type of carbon nanotubes 22 from the sample 20.

Additional, optional steps may be included in the processes including, but not limited to, washing the sample 20 and bound molecules 26, 27, 28 to ensure that only the proper nanotube 22, 23, 24 is bound to the proper biomolecule 26, 27, 28 and allowing molecules 26, 27, 28 that do not have a binding biomolecule present to flow through and exit the apparatus.

The embodiments herein allow for large-scale, non-chemical based separation of nanotube populations and allow manufacturers of various types of products to provide stronger and lighter products. Examples of the types of manufacturers/products that could utilize the embodiments herein include, but are not limited to, vehicles (cars, trains, tanks, airplanes, submarines, aerospace, etc.), weapons (guns, rockets, bullets, etc.), centrifuge rotors, appliances, tools, and batteries to name just a few.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method of purifying nanotubes, said method comprising:
providing a sample of heterogeneous nanotubes comprising at least two different types of carbon nanotubes comprising a first type of carbon nanotubes and a second type of carbon nanotubes;
providing a plurality of first type of molecules;
introducing said sample of heterogeneous nanotubes to said plurality of first type of molecules;
binding said first type of carbon nanotubes to said plurality of first type of molecules; and
separating said first type of carbon nanotubes from said sample.

2. The method of claim 1, further comprising:
providing a plurality of second type of molecules;
introducing said sample of heterogeneous nanotubes to said plurality of second type of molecules;
binding said second type of carbon nanotubes to said plurality of second type of molecules; and
separating said second type of carbon nanotubes from said sample.

3. The method of claim 2, wherein said at least two different types of carbon nanotubes comprises a third type of carbon nanotubes.

4. The method of claim 3, further comprising:
providing a plurality of third type of molecules;
introducing said sample of heterogeneous nanotubes to said plurality of third type of molecules;
binding said third type of carbon nanotubes to said plurality of third type of molecules; and
separating said third type of carbon nanotubes from said sample.

5. The method of claim 1, further comprising washing said sample of heterogeneous nanotubes.

6. The method of claim 4, further comprising washing said first type of molecules, said second type of molecules, and said third type of molecules.

7. The method of claim 4, wherein any of said first type of molecules, said second type of molecules, and said third type of molecules comprises any of a peptide, protein, amino acid, nucleic acid, deoxyribonucleic acid, ribonucleic acid, and peptide nucleic acid.

8. The method of claim 4, wherein said first type of carbon nanotubes, said second type of carbon nanotubes, and said third type of carbon nanotubes are each less than approximately 40 microns in length.

9. A method of separating different types of nanotubes from one another using non-chemical processing, said method comprising:
providing a sample of heterogeneous nanotubes comprising a first type of carbon nanotube and a second type of carbon nanotube;
providing a first type of molecule;
introducing said first type of molecule to said sample of heterogeneous nanotubes;
binding said first type of molecule to said first type of carbon nanotube; and
separating said first type of carbon nanotube from said sample.

10. The method of claim 9, further comprising:
providing a second type of molecule;
introducing said second type of molecule to said sample of heterogeneous nanotubes;
binding said second type of molecule to said second type of carbon nanotube; and
separating said second type of carbon nanotube from said sample.

11. The method of claim 10, wherein said sample of heterogeneous nanotubes comprises a third type of carbon nanotube.

12. The method of claim 11, further comprising:
providing a third type of molecule;
introducing said third type of molecule to said sample of heterogeneous nanotubes;
binding said third type of molecule to said third type of carbond nanotube; and
separating said third type of carbon nanotube from said sample.

13. The method of claim 12, further comprising washing any of said sample of heterogeneous nanotubes, and said first type of molecule, said second type of molecule, and said third type of molecule.

14. The method of claim 12, wherein any of said first type of molecule, said second type of molecule, and said third type of molecule comprises any of a peptide, protein, amino acid, nucleic acid, deoxyribonucleic acid, ribonucleic acid, and peptide nucleic acid, and wherein said first type of carbon nanotube, said second type of carbon nanotube, and said third type of carbon nanotube are each less than approximately 40 microns in length.

15. An apparatus for purifying nanotubes, said apparatus comprising:
a sample of heterogeneous nanotubes comprising at least two different types of carbon nanotubes comprising a first type of carbon nanotubes and a second type of carbon nanotubes;
a purification column comprising at least one solid support structure comprising a plurality of first type of molecules;
a buffer flow zone in said purification column that allows said sample of heterogeneous nanotubes to interact with said plurality of first type of molecules, wherein the interaction comprises (i) binding said first type of carbon nanotubes to said plurality of first type of molecules, and (ii) separating said first type of carbon nanotubes from said sample; and
a first collection unit that collects the separated said first type of carbon nanotubes.

16. The apparatus of claim 15, wherein said at least one solid support structure comprises a first solid support structure comprising said plurality of first type of molecules, wherein said apparatus further comprises a second solid support structure comprising a plurality of second type of molecules, wherein said buffer flow zone allows said sample of heterogeneous nanotubes to interact with said plurality of second type of molecules, wherein the interaction comprises (iii) binding said second type of carbon nanotubes to said plurality of second type of molecules, and (iv) separating said second type of carbon nanotubes from said sample, and wherein said apparatus further comprises a second collection unit that collects the separated said second type of carbon nanotubes.

17. The apparatus of claim 16, wherein said at least two different types of carbon nanotubes comprises a third type of carbon nanotubes.

18. The apparatus of claim 17, further comprising a third solid support structure comprising a plurality of third type of molecules, wherein said buffer flow zone allows said sample of heterogeneous nanotubes to interact with said plurality of third type of molecules, wherein the interaction comprises (v) binding said third type of carbon nanotubes to said plurality of third type of molecules, and (vi) separating said third type of carbon nanotubes from said sample, and wherein said apparatus further comprises a third collection unit that collects the separated said third type of carbon nanotubes.

19. The apparatus of claim 18, wherein any of said first type of molecules, said second type of molecules, and said third type of molecules comprises any of a peptide, protein, amino acid, nucleic acid, deoxyribonucleic acid, ribonucleic acid, and peptide nucleic acid.

20. The apparatus of claim 19, wherein said first type of carbon nanotubes, said second type of carbon nanotubes, and said third type of carbon nanotubes are each less than approximately 40 microns in length.
